(19) United States
(12) Patent Application Publication
(10) Pub. No.: US 2025/0095191 A1
(43) Pub. Date: Mar. 20, 2025

(54) IMAGE PROCESSING DEVICE, DISPLAY DEVICE, ENDOSCOPE DEVICE, IMAGE PROCESSING METHOD, IMAGE PROCESSING PROGRAM, TRAINED MODEL, TRAINED MODEL GENERATION METHOD, AND TRAINED MODEL GENERATION PROGRAM

(71) Applicant: FUJIFILM CORPORATION, Tokyo (JP)

(72) Inventor: Masaaki OOSAKE, Kanagawa (JP)

(21) Appl. No.: 18/970,897

(22) Filed: Dec. 6, 2024

Related U.S. Application Data
(63) Continuation of application No. PCT/JP2023/016141, filed on Apr. 24, 2023.

(30) Foreign Application Priority Data
Jul. 19, 2022 (JP) 2022-115110

(51) Int. Cl. G06T 7/70; A61B 1/00; G06T 7/11

(52) U.S. Cl. CPC G06T 7/70; A61B 1/000096; G06T 7/11; G06T 2207/10068; G06T 2207/20081; G06T 2207/30028

(57) ABSTRACT

An image processing device includes a processor, in which the processor acquires a lumen direction that is a direction in which an endoscope is inserted, from an image obtained by imaging a tubular organ via a camera provided in the endoscope, in accordance with a trained model obtained through machine learning based on a positional relationship between a plurality of division regions obtained by dividing the image and a lumen corresponding region included in the image, and outputs lumen direction information that is information indicating the lumen direction.

### CROSS-REFERENCE TO RELATED APPLICATIONS

This application is a continuation application of International Application No. PCT/JP2023/016141, filed Apr. 24, 2023, the disclosure of which is incorporated herein by reference in its entirety. Further, this application claims priority from Japanese Patent Application No. 2022-115110, filed Jul. 19, 2022, the disclosure of which is incorporated herein by reference in its entirety.

### BACKGROUND

### 1. Technical Field

The technology of the present disclosure relates to an image processing device, a display device, an endoscope device, an image processing method, an image processing program, a trained model, a trained model generation method, and a trained model generation program.

### 2. Description of the Related Art

JP4077716B discloses an endoscope insertion direction detection device. An endoscope insertion direction detection device comprises an image input unit that inputs an endoscopic image from an endoscope inserted into a body cavity, a pixel extraction unit that extracts a pixel having a predetermined density value from the endoscopic image input by the image input unit or that extracts a pixel having a gradient of a rate of change of the density value with a neighboring pixel among pixels forming the endoscopic image which is a predetermined value, a region shape estimation unit that obtains a shape of a specific region composed of the pixels extracted by the pixel extraction unit, and an insertion direction determination unit that determines an insertion direction of the endoscope into the body cavity from the shape of the specific region obtained by the region shape estimation unit.

JP5687583B discloses an endoscope insertion direction detection method. An endoscope insertion direction detection method comprises a first step of inputting an endoscopic image, a first detection step of executing processing for detecting an insertion direction of an endoscope based on any one of a gradient of brightness in the endoscopic image, a shape of halation in the endoscopic image, or a movement of a visual field of the endoscopic image, based on the endoscopic image, a determination step of determining whether or not the insertion direction of the endoscope is detected by the first detection step, and a second detection step of executing processing, which is different from the processing in the first detection step, for detecting the insertion direction of the endoscope based on any one of the gradient of brightness in the endoscopic image, the shape of halation in the endoscopic image, or the movement of the visual field of the endoscopic image, which is different from the first detection step, based on the endoscopic image in a case in which it is determined in the determination step that the insertion direction of the endoscope is not detected.

### SUMMARY

One embodiment according to the technology of the present disclosure provides an image processing device, a display device, an endoscope device, an image processing method, an image processing program, a trained model, a trained model generation method, and a trained model generation program that implement output of accurate lumen direction information.

A first aspect according to the technology of the present disclosure relates to an image processing device comprising: a processor, in which the processor acquires a lumen direction that is a direction in which an endoscope is inserted, from an image obtained by imaging a tubular organ via a camera provided in the endoscope, in accordance with a trained model obtained through machine learning based on a positional relationship between a plurality of division regions obtained by dividing the image and a lumen corresponding region included in the image, and outputs lumen direction information that is information indicating the lumen direction.

A second aspect according to the technology of the present disclosure relates to the image processing device according to the first aspect, in which the lumen corresponding region is a region in a predetermined range including a lumen region in the image.

A third aspect according to the technology of the present disclosure relates to the image processing device according to the first aspect, in which the lumen corresponding region is an end part of an observation range of the camera in a direction in which a position of the lumen region is estimated from a fold region in the image.

A fourth aspect according to the technology of the present disclosure relates to the image processing device according to any one of the first to third aspects, in which a direction of a division region overlapping the lumen corresponding region among the plurality of division regions is the lumen direction.

A fifth aspect according to the technology of the present disclosure relates to the image processing device according to any one of the first to fourth aspects, in which the trained model is a data structure configured to cause the processor to estimate a position of the lumen region based on a shape and/or an orientation of a fold region in the image.

A sixth aspect according to the technology of the present disclosure relates to the image processing device according to any one of the first to fifth aspects, in which the lumen direction is a direction in which a division region having a largest area overlapping the lumen corresponding region in the image among the plurality of division regions is present.

A seventh aspect according to the technology of the present disclosure relates to the image processing device according to any one of the first to fifth aspects, in which the lumen direction is a direction in which, among the plurality of division regions, a first division region that is a division region having a largest area overlapping the lumen corresponding region in the image is present and a direction in which a second division region that is a division region having a second largest area overlapping the lumen corresponding region following the first division region is present.

An eighth aspect according to the technology of the present disclosure relates to the image processing device according to any one of the first to seventh aspects, in which the division regions include a central region of the image and a plurality of radial regions that are present radially from the central region toward an outer edge of the image.

A ninth aspect according to the technology of the present disclosure relates to the image processing device according to the eighth aspect, in which eight radial regions are present radially.

A tenth aspect according to the technology of the present disclosure relates to the image processing device according to any one of the first to seventh aspects, in which the division regions include a central region of the image and a plurality of peripheral regions present on an outer edge side of the image with respect to the central region.

An eleventh aspect according to the technology of the present disclosure relates to the image processing device according to any one of the first to seventh aspects, in which the division regions are obtained by dividing the image into regions in three or more directions toward an outer edge of the image with a center of the image as a starting point.

A twelfth aspect according to the technology of the present disclosure relates to the image processing device according to any one of the first to seventh aspects, in which the division regions include a central region of the image and a plurality of peripheral regions present on an outer edge side of the image with respect to the central region, and the peripheral regions are obtained by dividing the outer edge side of the image with respect to the central region in three or more directions from the central region toward an outer edge of the image.

A thirteenth aspect according to the technology of the present disclosure relates to a display device that displays information corresponding to the lumen direction information output by the processor of the image processing device according to any one of the first to twelfth aspects.

A fourteenth aspect according to the technology of the present disclosure relates to an endoscope device comprising: the image processing device according to any one of the first to twelfth aspects; and the endoscope.

A fifteenth aspect according to the technology of the present disclosure relates to an image processing method comprising: acquiring a lumen direction that is a direction in which an endoscope is inserted, from an image obtained by imaging a tubular organ via a camera provided in the endoscope, in accordance with a trained model obtained through machine learning based on a positional relationship between a plurality of division regions obtained by dividing the image and a lumen corresponding region included in the image; and outputting lumen direction information that is information indicating the lumen direction.

A sixteenth aspect according to the technology of the present disclosure relates to an image processing program for causing a first computer to execute image processing comprising: acquiring a lumen direction that is a direction in which an endoscope is inserted, from an image obtained by imaging a tubular organ via a camera provided in the endoscope, in accordance with a trained model obtained through machine learning based on a positional relationship between a plurality of division regions obtained by dividing the image and a lumen corresponding region included in the image; and outputting lumen direction information that is information indicating the lumen direction.

A seventeenth aspect according to the technology of the present disclosure relates to a trained model that is obtained through machine learning based on a positional relationship between a plurality of division regions obtained by dividing an image obtained by imaging a tubular organ via a camera provided in an endoscope and a lumen corresponding region included in the image.

An eighteenth aspect according to the technology of the present disclosure relates to a trained model generation method comprising: acquiring an image obtained by imaging a tubular organ via a camera provided in an endoscope; and executing, on a model, machine learning based on a positional relationship between a plurality of division regions obtained by dividing the image and a lumen corresponding region included in the image.

A nineteenth aspect according to the technology of the present disclosure relates to a trained model generation program for causing a second computer to execute trained model generation processing comprising: acquiring an image obtained by imaging a tubular organ via a camera provided in an endoscope; and executing, on a model, machine learning based on a positional relationship between a plurality of division regions obtained by dividing the image and a lumen corresponding region included in the image.

### BRIEF DESCRIPTION OF THE DRAWINGS

Exemplary embodiments according to the technique of the present disclosure will be described in detail based on the following figures, wherein:

FIG. 1 is a conceptual diagram showing an example of an aspect in which an endoscope system is used;

FIG. 2 is a conceptual diagram showing an example of an overall configuration of the endoscope system;

FIG. 3 is a block diagram showing an example of a hardware configuration of an endoscope device;

FIG. 4 is a block diagram showing an example of a configuration of the endoscope device;

FIG. 5 is a block diagram showing an example of a hardware configuration of an information processing device;

FIG. 6 is a conceptual diagram showing an example of processing contents of an operation unit of the information processing device;

FIG. 7 is a conceptual diagram showing an example of processing contents of the operation unit of the information processing device;

FIG. 8 is a conceptual diagram showing an example of processing contents of a training data generation unit and a learning execution unit of the information processing device;

FIG. 9 is a conceptual diagram showing an example of processing contents of a lumen direction estimation unit of a control device;

FIG. 10 is a conceptual diagram showing an example of processing contents of the lumen direction estimation unit, an information generation unit, and a display control unit of the control device;

FIG. 11 is a conceptual diagram showing an example of processing contents of the lumen direction estimation unit, the information generation unit, and the display control unit of the control device;

FIG. 12 is a flowchart showing an example of a machine learning processing flow;

FIG. 13 is a flowchart showing an example of an endoscopic image processing flow;

FIG. 14 is a conceptual diagram showing an example of processing contents of an operation unit according to a first modification example;

FIG. 15 is a conceptual diagram showing an example of processing contents of a lumen direction estimation unit according to the first modification example;

FIG. 16 is a conceptual diagram showing an example of processing contents of the lumen direction estimation unit, an information generation unit, and a display control unit according to the first modification example;

FIG. 17 is a conceptual diagram showing an example of processing contents of the lumen direction estimation unit according to the first modification example;

FIG. 18 is a conceptual diagram showing an example of processing contents of an operation unit according to a second modification example; and

FIG. 19 is a conceptual diagram showing an example of processing contents of an operation unit according to a third modification example.

### DETAILED DESCRIPTION

Hereinafter, an example of embodiments of an image processing device, a display device, an endoscope device, an image processing method, an image processing program, a trained model, a trained model generation method, and a trained model generation program according to the technology of the present disclosure will be described based on the accompanying drawings.

The terms used in the following description will be described first.

CPU is an abbreviation for “central processing unit”. GPU is an abbreviation for “graphics processing unit”. RAM is an abbreviation for “random-access memory”. NVM is an abbreviation for “non-volatile memory”. EEPROM is an abbreviation for “electrically erasable programmable read-only memory”. ASIC is an abbreviation for “application-specific integrated circuit”. PLD is an abbreviation for “programmable logic device”. FPGA is an abbreviation for “field-programmable gate array”. SoC is an abbreviation for “system-on-a-chip”. SSD is an abbreviation for “solid-state drive”. USB is an abbreviation for “Universal Serial Bus”. HDD is an abbreviation for “hard disk drive”. EL is an abbreviation for “electro-luminescence”. CMOS is an abbreviation for “complementary metal-oxide-semiconductor”. CCD is an abbreviation for “charge-coupled device”. BLI is an abbreviation for “blue light imaging”. LCI is an abbreviation for “linked color imaging”. CNN is an abbreviation for “convolutional neural network”. AI is an abbreviation for “artificial intelligence”.

### First Embodiment

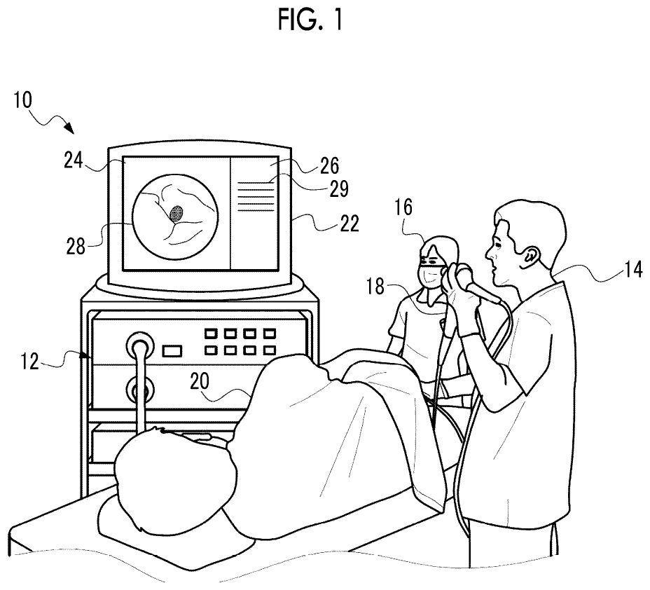

As shown in FIG. 1 as an example, an endoscope system 10 comprises an endoscope device 12. The endoscope device 12 is used by a doctor 14 in an endoscopy. In addition, at least one assistant staff member 16 (for example, a nurse) assists the doctor 14 who executes the endoscopy. In the following description, in a case in which it is not necessary to distinguish between the doctor 14 and the assistant staff member 16, the doctor 14 and the assistant staff member 16 are also referred to as a “user” without reference numerals.

The endoscope device 12 comprises an endoscope 18, and is a device for executing medical care for an inside of a body of a subject 20 (for example, a patient) via the endoscope 18. The endoscope device 12 is an example of an “endoscope device” according to the technology of the present disclosure.

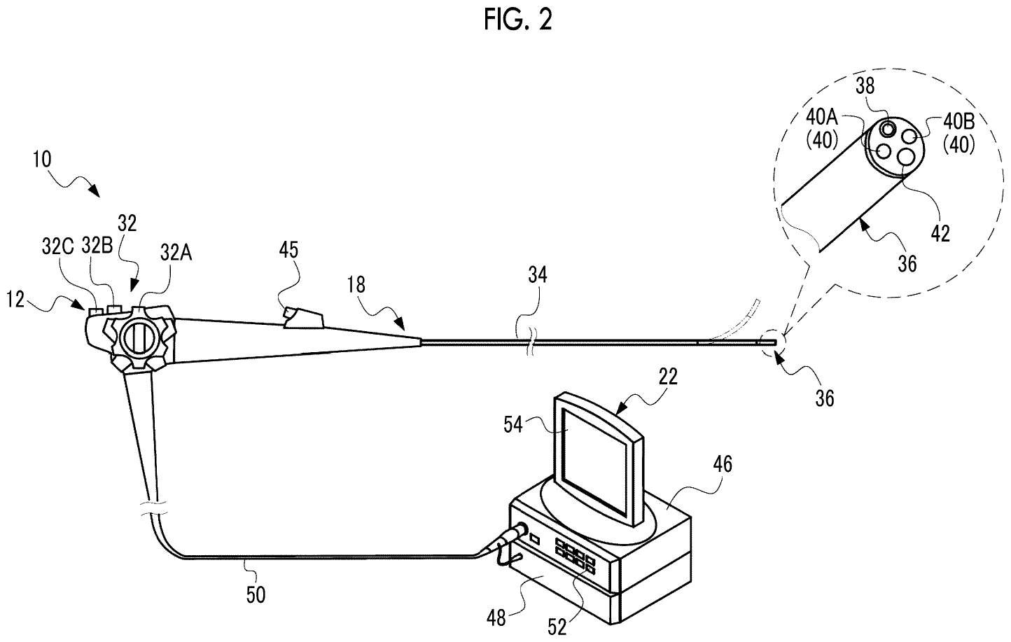

The endoscope 18 acquires an image showing an aspect of the inside of the body by imaging the inside of the body of the subject 20 via a camera 38 (see FIG. 2) described below. Then, the endoscope 18 outputs the image showing the aspect of the inside of the body. In the example shown in FIG. 1, an aspect is shown in which the endoscope 18 is inserted into a body cavity from an anus of the subject 20. It should be noted that, in the example shown in FIG. 1, the endoscope 18 is inserted into the body cavity from the anus of the subject 20, but this is merely an example, and the endoscope 18 may be inserted into the body cavity from a mouth, a nostril, a perforation, or the like of the subject 20, and a location into which the endoscope 18 is inserted is determined by a type of the endoscope 18, a surgical method using the endoscope 18, and the like.

A display device 22 displays various kinds of information including the image. Examples of the display device 22 include a liquid-crystal display and an EL display. The display device 22 displays a plurality of screens side by side. In the example shown in FIG. 1, screens 24 and 26 are shown as examples of the plurality of screens. The display device 22 is an example of a “display device” according to the technology of the present disclosure.

An endoscopic image 28 is displayed on the screen 24. The endoscopic image 28 is an image acquired by imaging an observation target region via the camera 38 (see FIG. 2) provided in the endoscope 18 in the body cavity of the subject 20. The observation target region includes an interior wall of a large intestine. The interior wall of the large intestine is merely an example, and may be an interior wall or an exterior wall of another part such as a small intestine, a duodenum, or a stomach.

The endoscopic image 28 displayed on the screen 24 is one frame included in a moving image including a plurality of frames. That is, the endoscopic images 28 of the plurality of frames are displayed on the screen 24 at a predetermined frame rate (for example, 30 frames/second or 60 frames/second).

On the screen 26, for example, subject specification information 29 is displayed. The subject specification information 29 is information related to the subject 20. The subject specification information 29 includes, for example, a name of the subject 20, an age of the subject 20, and an identification number for identifying the subject 20.

As shown in FIG. 2 as an example, the endoscope 18 comprises an operating part 32 and an insertion part 34. The operating part 32 comprises a rotation operation knob 32A, an air/water supply button 32B, and a suction button 32C. The insertion part 34 is formed in a tubular shape. An outer contour of the insertion part 34 in a cross-sectional view has a circular shape. The rotation operation knob 32A of the operating part 32 is operated to partially bend the insertion part 34 or to rotate the insertion part 34 about an axis of the insertion part 34. As a result, the insertion part 34 is moved to a back side of the body while being bent in accordance with a shape of the inside of the body (for example, a shape of a tubular organ) or while being rotated about the axis of the insertion part 34 in accordance with an internal part of the body. In addition, the air/water supply button 32B is operated to supply water or air into the body from a distal end part 36, and the suction button 32C is operated to suction water or air in the body.

The camera 38, an illumination device 40, and a treatment tool opening 42 are provided at the distal end part 36. The camera 38 images the inside of the tubular organ by using an optical method. Examples of the camera 38 include a CMOS camera. However, this is merely an example, and another type of camera such as a CCD camera may be adopted. The camera 38 is an example of a “camera” according to the technology of the present disclosure.

The illumination device 40 includes an illumination window 40A and an illumination window 40B. The illumination device 40 emits light via the illumination window 40A and the illumination window 40B. Examples of a type of light emitted from the illumination device 40 include visible light (for example, white light), invisible light (for example, near-infrared light), and/or special light. Examples of the special light include light for BLI and/or light for LCI.

The treatment tool opening 42 is an opening through which a treatment tool protrudes from the distal end part 36. The treatment tool opening 42 also functions as a suction port for suctioning blood, internal waste, and the like. The treatment tool is inserted into the insertion part 34 from a treatment tool insertion port 45. The treatment tool passes through the insertion part 34 and protrudes outward from the treatment tool opening 42. Examples of the treatment tool include a puncture needle, a wire, a scalpel, gripping forceps, a guide sheath, and an ultrasound probe.

The endoscope device 12 comprises a control device 46 and a light source device 48. The endoscope 18 is connected to the control device 46 and the light source device 48 via a cable 50. The control device 46 is a device that controls the entire endoscope device 12. The light source device 48 is a device that emits light under the control of the control device 46 to supply the light to the illumination device 40.

The control device 46 is provided with a plurality of hard keys 52. The plurality of hard keys 52 receive instructions from the user. A touch panel 54 is provided on the screen of the display device 22. The touch panel 54 is electrically connected to the control device 46 to receive the instructions from the user. The display device 22 is also electrically connected to the control device 46.

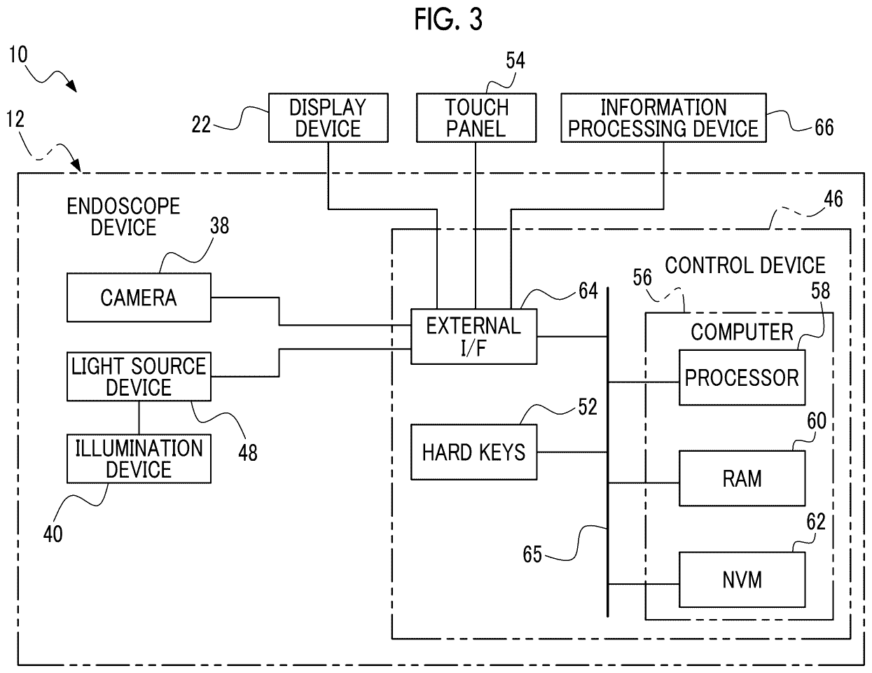

As shown in FIG. 3 as an example, the control device 46 comprises a computer 56. The computer 56 is an example of an “image processing device” and a “first computer” according to the technology of the present disclosure. The computer 56 comprises a processor 58, a RAM 60, and an NVM 62, and the processor 58, the RAM 60, and the NVM 62 are electrically connected to each other. The processor 58 is an example of a “processor” according to the technology of the present disclosure.

The control device 46 comprises the hard keys 52 and an external I/F 64. The hard keys 52, the processor 58, the RAM 60, the NVM 62, and the external I/F 64 are connected to a bus 65.

For example, the processor 58 includes a CPU and a GPU and controls the entire control device 46. The GPU operates under the control of the CPU and is responsible for execution of various kinds of graphics-related processing. It should be noted that the processor 58 may be one or more CPUs integrated with a GPU function, or may be one or more CPUs not integrated with the GPU function.

The RAM 60 is a memory in which information is stored temporarily, and is used as a work memory by the processor 58. The NVM 62 is a non-volatile storage device that stores various programs and various parameters. An example of the NVM 62 is a flash memory (for example, an EEPROM and/or an SSD). It should be noted that the flash memory is merely an example, and another non-volatile storage device such as an HDD or a combination of two or more types of non-volatile storage devices may be used.

The hard keys 52 receive the instructions from the user and output signals indicating the received instructions to the processor 58. Therefore, the instructions received by the hard keys 52 are recognized by the processor 58.

The external I/F 64 controls the exchange of various kinds of information between a device (hereinafter, also referred to as an “external device”) present outside the control device 46 and the processor 58. Examples of the external I/F 64 include a USB interface.

The endoscope 18 as one of the external devices is connected to the external I/F 64, and the external I/F 64 controls the exchange of various kinds of information between the endoscope 18 and the processor 58. The processor 58 controls the endoscope 18 via the external I/F 64. In addition, the processor 58 acquires, via the external I/F 64, the endoscopic image 28 (see FIG. 1) obtained by imaging the inside of the tubular organ via the camera 38.

The light source device 48 as one of the external devices is connected to the external I/F 64, and the external I/F 64 controls the exchange of various kinds of information between the light source device 48 and the processor 58. The light source device 48 supplies the light to the illumination device 40 under the control of the processor 58. The illumination device 40 emits the light supplied from the light source device 48.

The display device 22 as one of the external devices is connected to the external I/F 64, and the processor 58 controls the display device 22 via the external I/F 64, so that the display device 22 displays various kinds of information.

The touch panel 54 as one of the external devices is connected to the external I/F 64, and the processor 58 acquires the instruction received by the touch panel 54 via the external I/F 64.

An information processing device 66 is connected to the external I/F 64 as one of the external devices. Examples of the information processing device 66 include a server. It should be noted that the server is merely an example, and the information processing device 66 may be a personal computer.

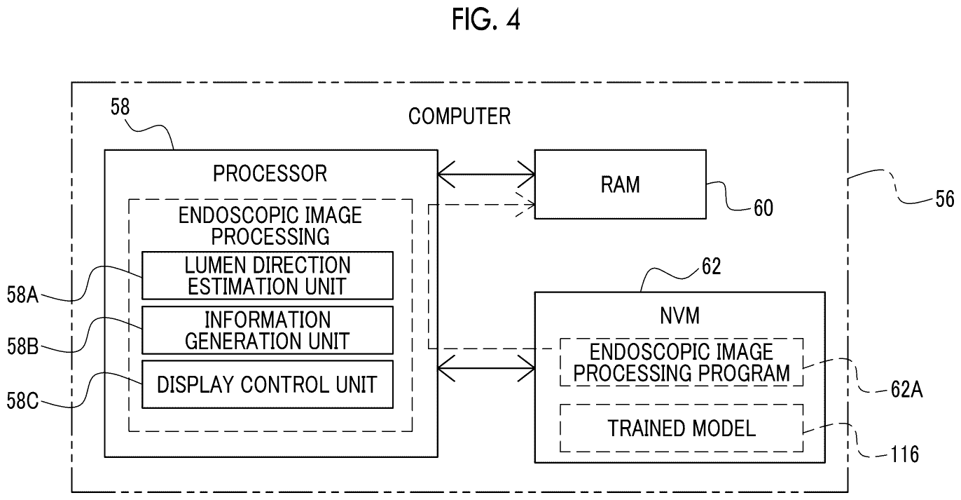

The external I/F 64 controls the exchange of various kinds of information between the information processing device 66 and the processor 58. The processor 58 requests the information processing device 66 to provide a service via the external I/F 64 or acquires a trained model 116 (see FIG. 4) from the information processing device 66 via the external I/F 64.

In a case in which the inside of the tubular organ (for example, the large intestine) in the body is observed by using the camera 38 provided in the endoscope 18, the endoscope 18 is inserted along a lumen. In such a case, it may be difficult for the user to recognize a lumen direction that is a direction in which the endoscope 18 is inserted. In addition, in a case in which the endoscope 18 is inserted in a direction different from the lumen direction, the endoscope 18 hits the interior wall of the tubular organ, which imposes an unnecessary burden on the subject 20 (for example, the patient).

Therefore, in view of such circumstances, in the present embodiment, endoscopic image processing is executed by the processor 58 of the control device 46. As shown in FIG. 4 as an example, the NVM 62 stores an endoscopic image processing program 62A. The processor 58 reads out the endoscopic image processing program 62A from the NVM 62 to execute the readout endoscopic image processing program 62A on the RAM 60. The endoscopic image processing is implemented by the processor 58 operating as a lumen direction estimation unit 58A, an information generation unit 58B, and a display control unit 58C in accordance with the endoscopic image processing program 62A executed on the RAM 60.

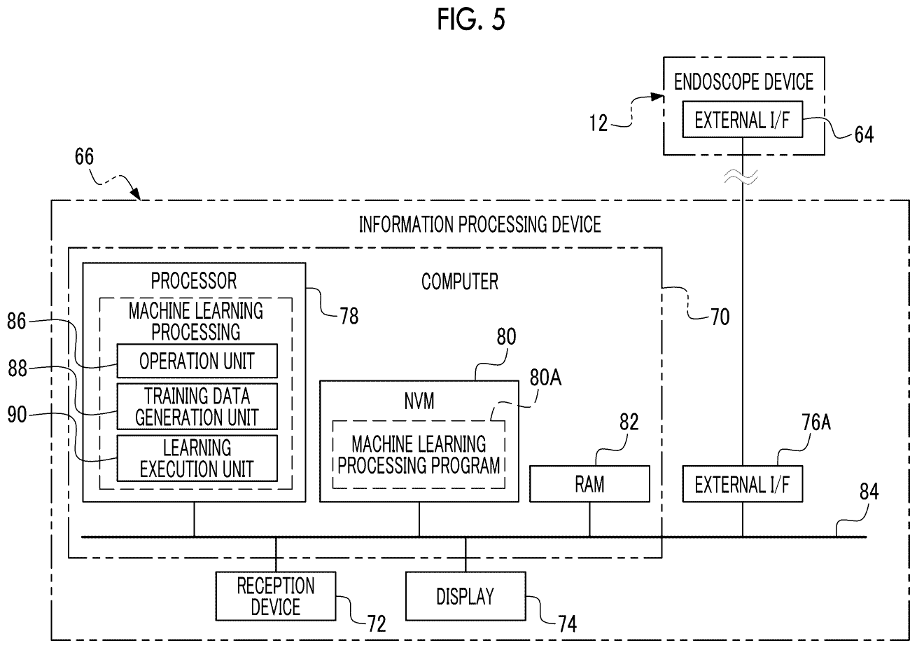

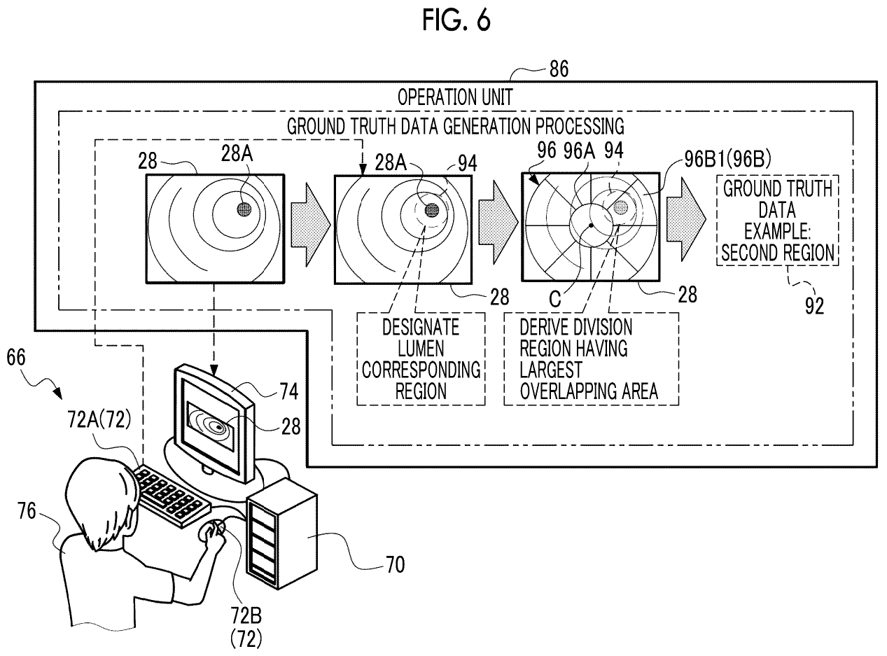

As shown in FIG. 5 as an example, machine learning processing is executed by a processor 78 (see FIG. 5) of the information processing device 66. The information processing device 66 is a device used in the machine learning. The information processing device 66 is used by an annotator 76 (see FIG. 6). The annotator 76 means an operator (that is, an operator who performs labeling) who assigns an annotation for machine learning to given data.

The information processing device 66 comprises a computer 70, a reception device 72, a display 74, and an external I/F 76A. The computer 70 is an example of a “second computer” according to the technology of the present disclosure.

The computer 70 comprises a processor 78, an NVM 80, and an RAM 82. The processor 78, the NVM 80, and the RAM 82 are connected to a bus 84. In addition, the reception device 72, the display 74, and the external I/F 76 are also connected to the bus 84.

The processor 78 controls the entire information processing device 66. The processor 78, the NVM 80, and the RAM 82 are the same hardware resources as the processor 58, the NVM 62, and the RAM 60.

The reception device 72 receives an instruction from the annotator 76. The processor 78 operates in response to the instructions received by the reception device 72.

The external I/F 76A is the same hardware resource as the external I/F 64. The external I/F 76A is connected to the external I/F 64 of the endoscope device 12 to control the exchange of various kinds of information between the endoscope device 12 and the processor 78.

The NVM 80 stores a machine learning processing program 80A. The processor 78 reads out the machine learning processing program 80A from the NVM 80 to execute the readout machine learning processing program 80A on the RAM 82. The processor 78 executes machine learning processing in accordance with the machine learning processing program 80A executed on the RAM 82. The machine learning processing is implemented by the processor 78 operating as an operation unit 86, a training data generation unit 88, and a learning execution unit 90 in accordance with the machine learning processing program 80A. The machine learning processing program 80A is an example of a “trained model generation program” according to the technology of the present disclosure.

As shown in FIG. 6 as an example, first, the operation unit 86 displays the endoscopic image 28 on the display 74. Here, the endoscopic image 28 is, for example, an image that is acquired in a past medical examination and/or treatment and that is stored in advance in the NVM 80, but this is merely an example. The endoscopic image 28 may be an image that is stored in an image server (not shown) as the external device and that is acquired via the external I/F 76A (see FIG. 5). In a state in which the endoscopic image 28 is displayed on the display 74, the annotator 76 designates a lumen corresponding region 94 in the endoscopic image 28 to the computer 70 via the reception device 72 (for example, a keyboard 72A and/or a mouse 72B). For example, the annotator 76 designates a lumen region 28A in the endoscopic image 28 displayed on the display 74 by using a pointer (not shown). Here, the lumen region 28A means an image region showing the lumen in the endoscopic image 28.

The operation unit 86 recognizes the lumen corresponding region 94 designated by the annotator 76 via the reception device 72. Here, the lumen corresponding region 94 means a region in a predetermined range (for example, a range of a radius of 64 pixels from the center of the lumen region 28A) including the lumen region 28A in the endoscopic image 28. The lumen corresponding region 94 is an example of a “lumen corresponding region” according to the technology of the present disclosure. In addition, a plurality of division regions 96 are obtained by virtually dividing the endoscopic image 28 via the operation unit 86. The division region 96 is an example of a “division region” according to the technology of the present disclosure. For example, the lumen corresponding region 94 is a region including the lumen region 28A in the endoscopic image 28 and having a size that can be inscribed in the division region 96 described later.

In the example shown in FIG. 6, the endoscopic image 28 is divided into a central region 96A and eight radial regions 96B. The central region 96A is, for example, a circular region centered on a center C in the endoscopic image 28. In addition, the radial region 96B is a region that is present radially from the central region 96A toward an outer edge of the endoscopic image 28. Here, although the eight radial regions 96B are shown, this is merely an example. For example, the number of radial regions 96B may be seven or less or may be nine or more. The central region 96A is an example of a “central region” according to the technology of the present disclosure, and the radial region 96B is an example of a “radial region” according to the technology of the present disclosure.

In the operation unit 86, a direction of the division region 96 overlapping the lumen corresponding region 94 among the plurality of division regions 96 is set as the lumen direction. Specifically, the operation unit 86 derives the division region 96 having a largest area overlapping the lumen corresponding region 94 among the plurality of division regions 96. For example, the operation unit 86 specifies a region in which each of the plurality of division regions 96 and the lumen corresponding region 94 overlap each other. The operation unit 86 calculates an area of the region in which the division region 96 and the lumen corresponding region 94 overlap each other. Then, the operation unit 86 specifies the division region 96 having a largest area of the region in which the division region 96 and the lumen corresponding region 94 overlap each other.

The operation unit 86 sets a direction of the division region 96 having the largest area overlapping the lumen corresponding region 94 as the lumen direction, and generates the division region 96 as ground truth data 92. In the example shown in FIG. 6, as an example of the ground truth data 92, a second region 96B1 of the radial region 96B is shown. The second region 96B1 is a region indicating the lumen direction (that is, a direction in which the camera 38 is inserted).

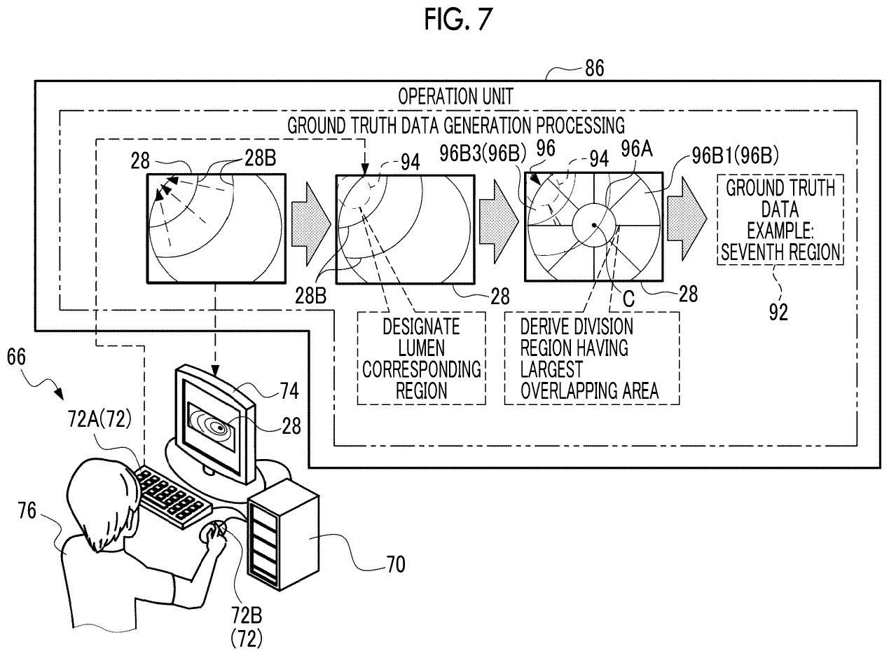

Here, although the form example has been described in which the lumen region 28A is captured in the endoscopic image 28, the technology of the present disclosure is not limited to this. For example, the lumen region 28A may not be captured in the endoscopic image 28. In such a case, as shown in FIG. 7 as an example, the annotator 76 estimates the lumen region 28A with reference to a position and/or a shape of a fold region 28B in the endoscopic image 28 displayed on the display 74. Here, the fold region 28B means an image region showing a fold in the tubular organ in the endoscopic image 28. Then, an end part of an observation range in the endoscopic image 28 is designated as the lumen corresponding region 94 by using the pointer (not shown).

The operation unit 86 derives the division region 96 having the largest area overlapping the lumen corresponding region 94 among the plurality of division regions 96. Then, the operation unit 86 generates the division region 96 having the largest area overlapping the lumen corresponding region 94 as the ground truth data 92. In the example shown in FIG. 7, as an example of the ground truth data 92, a seventh region 96B3 in the radial region 96B is shown. The seventh region 96B3 is a region indicating the lumen direction.

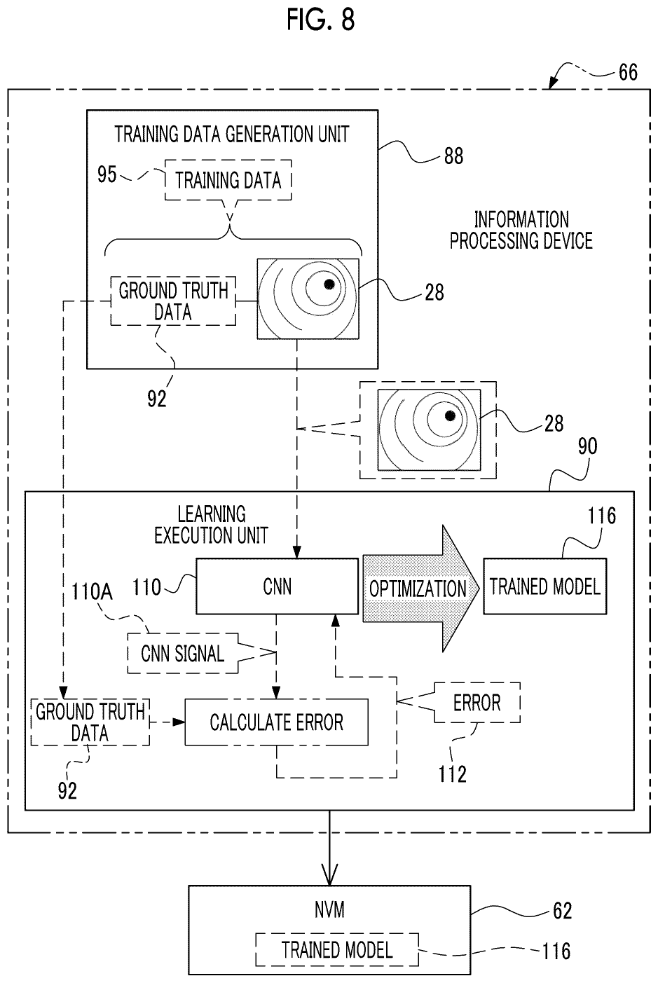

As shown in FIG. 8 as an example, the training data generation unit 88 acquires the endoscopic image 28 as an image for inference from the operation unit 86, and associates the ground truth data 92 with the acquired endoscopic image 28 to generate training data 95. The learning execution unit 90 acquires the training data 95 generated by the training data generation unit 88. The learning execution unit 90 executes the machine learning using the training data 95.

In the example shown in FIG. 8, the learning execution unit 90 includes a CNN 110. The learning execution unit 90 inputs the endoscopic image 28 included in the training data 95 to the CNN 110. It should be noted that, here, although the form example has been described in which the endoscopic images 28 are input to the CNN 110 one by one, the technology of the present disclosure is not limited to this. The endoscopic images 28 of the plurality of frames (for example, two to three frames) may be input to the CNN 110 at one time. In a case in which the endoscopic image 28 is input, the CNN 110 executes inference and outputs a CNN signal 110A indicating an inference result (for example, an image region predicted as the image region indicating the direction in which the lumen is present in all image regions constituting the endoscopic image 28). The learning execution unit 90 calculates an error 112 between the CNN signal 110A and the ground truth data 92 included in the training data 95.

The learning execution unit 90 optimizes the CNN 110 by adjusting a plurality of optimization variables in the CNN 110 so that the error 112 is minimized. Here, the plurality of optimization variables means, for example, a plurality of connection weights and a plurality of offset values included in the CNN 110.

The learning execution unit 90 repeatedly executes the learning processing of inputting the endoscopic image 28 to the CNN 110, calculating the error 112, and adjusting the plurality of optimization variables in the CNN 110, by using a plurality of pieces of training data 95. That is, the learning execution unit 90 optimizes the CNN 110 by adjusting the plurality of optimization variables in the CNN 110 so that the error 112 is minimized for each of a plurality of endoscopic images 28 included in the plurality of pieces of training data 95. A trained model 116 is generated by the CNN 110 in this manner. The trained model 116 is stored in a storage device by the learning execution unit 90. Examples of the storage device include the NVM 62 of the endoscope device 12, but this is merely an example. The storage device may be the NVM 80 of the information processing device 66. The trained model 116 stored in a predetermined storage device is used, for example, in lumen direction estimation processing in the endoscope device 12. The trained model 116 is an example of a “trained model” according to the technology of the present disclosure.

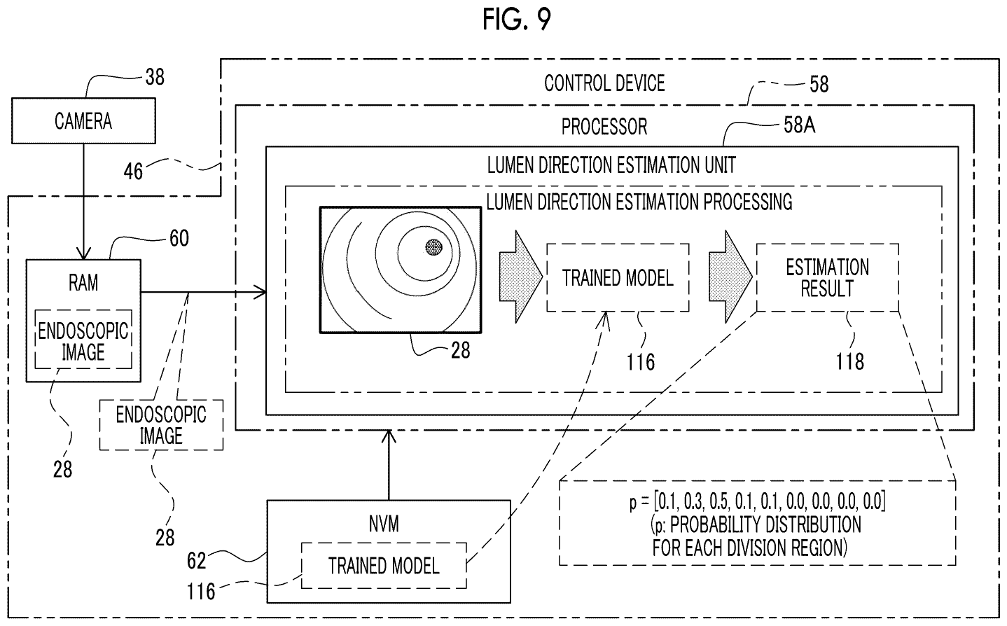

As shown in FIG. 9 as an example, in the endoscope device 12, the lumen direction estimation processing is executed by using the trained model 116 generated in the information processing device 66. First, the endoscopic image 28 is obtained by imaging the inside of the tubular organ in time series via the camera 38. The RAM 60 temporarily stores the endoscopic image 28. The lumen direction estimation unit 58A executes the lumen direction estimation processing based on the endoscopic image 28. In such a case, the lumen direction estimation unit 58A acquires the trained model 116 from the NVM 62. The lumen direction estimation unit 58A inputs the endoscopic image 28 to the trained model 116. In a case in which the endoscopic image 28 is input, the trained model 116 outputs an estimation result 118 in the lumen direction in the endoscopic image 28. The estimation result 118 is, for example, a probability that the lumen direction is present for each division region 96. A probability distribution p indicating nine probabilities corresponding to nine division regions 96 is output from the trained model 116 as the estimation result 118.

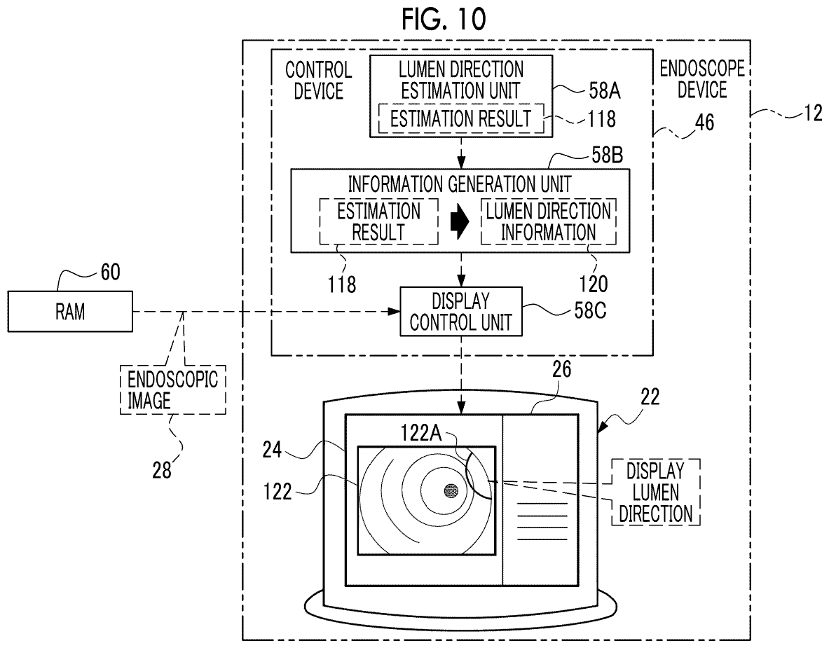

As shown in FIG. 10 as an example, the lumen direction estimation unit 58A outputs the estimation result 118 to the information generation unit 58B. The information generation unit 58B generates lumen direction information 120 based on the estimation result 118. The lumen direction information 120 is information indicating the lumen direction. The lumen direction information 120 is an example of “lumen direction information” according to the technology of the present disclosure. The information generation unit 58B generates the lumen direction information 120 by, for example, using the direction of the division region 96 indicating a highest probability value in the probability distribution p indicated by the estimation result 118 as the lumen direction. The information generation unit 58B outputs the lumen direction information 120 to the display control unit 58C.

The display control unit 58C acquires the endoscopic image 28 stored temporarily in the RAM 60. Further, the display control unit 58C generates an image 122 in which the lumen direction indicated by the lumen direction information 120 is superimposed and displayed on the endoscopic image 28. The display control unit 58C causes the display device 22 to display the image 122. In the example shown in FIG. 10, in the image 122, an arc 122A is shown on an outer periphery of the observation range of the endoscopic image 28 as display indicating the lumen direction.

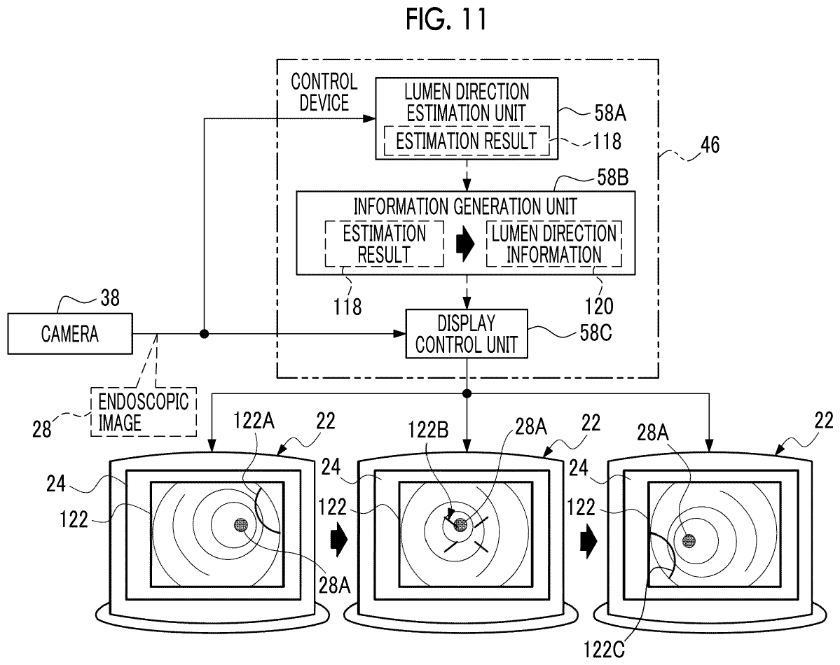

As shown in FIG. 11 as an example, the image 122 displayed on the display device 22 is updated each time the endoscopic image 28 is acquired. Specifically, the lumen direction estimation unit 58A executes the lumen direction estimation processing (see FIG. 10) each time the endoscopic image 28 is acquired from the camera 38. The lumen direction estimation unit 58A outputs the estimation result 118 obtained by the lumen direction estimation processing to the information generation unit 58B. The information generation unit 58B generates the lumen direction information 120 based on the estimation result 118. The display control unit 58C updates the image 122 on the display device 22 based on the lumen direction information 120 and the endoscopic image 28 acquired from the camera 38. As a result, in the image 122, the display indicating the lumen direction is changed in accordance with the lumen direction in the endoscopic image 28. In the example shown in FIG. 11, the lumen direction is moved in an order of the left side, the center, and the right side as viewed from a paper surface side in the endoscopic image 28. In the example shown in FIG. 11, in the image 122, the image 122 is updated in an order of the arc 122A, an X-shaped display 122B, and an arc 122C as the display indicating the lumen direction. It should be noted that, here, although the form example has been described in which the arcs 122A and 122C and the X-shaped display 122B are used as the display indicating the lumen direction, the technology of the present disclosure is not limited to this. For example, a symbol such as an arrow or text such as “upper right” may be used as the display indicating the lumen direction. Instead of or in addition to the display indicating the lumen direction via the display device 22, notification indicating the lumen direction via voice may be issued.

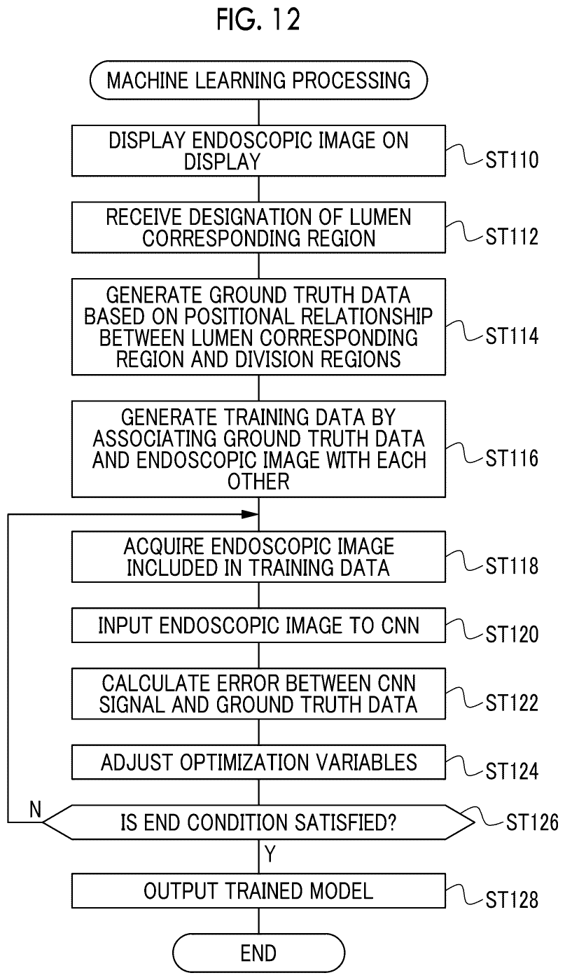

Next, an operation of the information processing device 66 will be described with reference to FIG. 12. FIG. 12 shows an example of a machine learning processing flow executed by the processor 78. The machine learning processing flow shown in FIG. 12 is an example of a “trained model generation method” according to the technology of the present disclosure.

In the machine learning processing shown in FIG. 12, first, in step ST110, the operation unit 86 displays the endoscopic image 28 on the display 74. After the execution of the processing of step ST110, the machine learning processing proceeds to step ST112.

In step ST112, the operation unit 86 receives the designation of the lumen corresponding region 94 input by the annotator 76 via the reception device 72 for the endoscopic image 28 displayed on the display 74 in step ST110. After the execution of the processing of step ST112, the machine learning processing proceeds to step ST114.

In step ST114, the operation unit 86 generates the ground truth data 92 based on a positional relationship between the lumen corresponding region 94 received in step ST112 and the division regions 96. After the execution of the processing of step ST114, the machine learning processing proceeds to step ST116.

In step ST116, the training data generation unit 88 generates the training data 95 by associating the ground truth data 92 generated in step ST114 and the endoscopic image 28 with each other. After the execution of the processing of step ST116, the machine learning processing proceeds to step ST118.

In step ST118, the learning execution unit 90 acquires the endoscopic image 28 included in the training data 95 generated in step ST116. After the execution of the processing of step ST118, the machine learning processing proceeds to step ST120.

In step ST120, the learning execution unit 90 inputs the endoscopic image 28 acquired in step ST118 to the CNN 110. After the execution of the processing of step ST120, the machine learning processing proceeds to step ST122.

In step ST122, the learning execution unit 90 calculates the error 112 by comparing the CNN signal 110A obtained by inputting the endoscopic image 28 to the CNN 110 in step ST120 with the ground truth data 92 associated with the endoscopic image 28. After the execution of the processing of step ST122, the machine learning processing proceeds to step ST124.

In step ST124, the learning execution unit 90 adjusts the optimization variables of the CNN 110 so that the error 112 calculated in step ST122 is minimized. After the execution of the processing of step ST124, the machine learning processing proceeds to step ST126.

In step ST126, the learning execution unit 90 determines whether or not a condition for ending the machine learning (hereinafter, referred to as an “end condition”) is satisfied. Examples of the end condition include a condition in which the error 112 calculated in step ST124 is equal to or less than a threshold value. In step ST126, in a case in which the end condition is not satisfied, a negative determination is made, and the machine learning processing proceeds to step ST118. In step ST126, in a case in which the end condition is satisfied, an affirmative determination is made, and the machine learning processing proceeds to step ST128.

In step ST128, the learning execution unit 90 outputs the trained model 116, which is the CNN 110 for which the machine learning has ended, to the outside (for example, to the NVM 62 of the endoscope device 12). After the execution of the processing of step ST128, the machine learning processing ends.

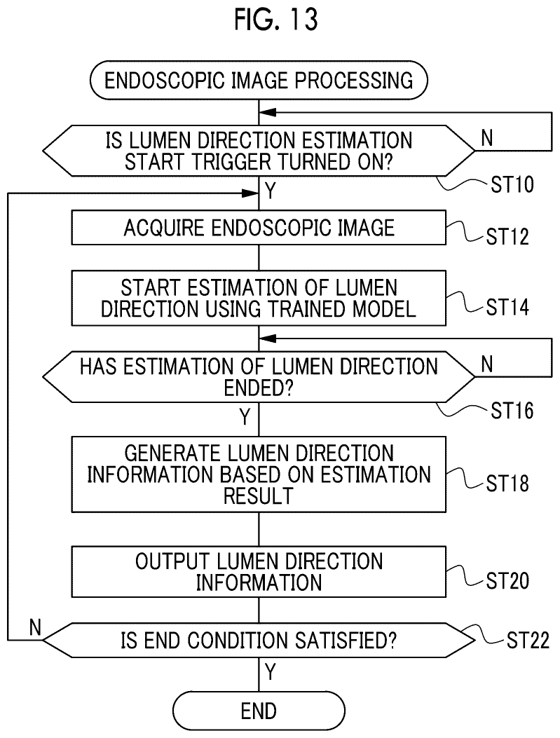

Next, an operation of the endoscope device 12 will be described with reference to FIG. 13. FIG. 13 shows an example of an endoscopic image processing flow executed by the processor 58. The endoscopic image processing flow shown in FIG. 13 is an example of an “image processing method” according to the technology of the present disclosure.

In the endoscopic image processing shown in FIG. 13, first, in step ST10, the lumen direction estimation unit 58A determines whether or not a lumen direction estimation start trigger is turned on. Examples of the lumen direction estimation start trigger include whether or not a lumen direction estimation start instruction (for example, an operation of a button (not shown) provided in the endoscope 18) issued by the user is received. In a case in which the lumen direction estimation start trigger is not turned on in step ST10, a negative determination is made, and the endoscopic image processing returns to step ST10 again. In a case in which the lumen direction estimation start trigger is turned on in step ST10, an affirmative determination is made, and the endoscopic image processing proceeds to step ST12. It should be noted that, in step ST10, the form example has been described in which it is determined whether or not the lumen direction estimation start trigger is turned on, but the technology of the present disclosure is not limited to this. The technology of the present disclosure is established even in an aspect in which the determination in step ST10 is omitted and the lumen direction estimation processing is always executed.

In step ST12, the lumen direction estimation unit 58A acquires the endoscopic image 28 from the RAM 60. After the execution of the processing of step ST12, the endoscopic image processing proceeds to step ST14.

In step ST14, the lumen direction estimation unit 58A starts the estimation of the lumen direction in the endoscopic image 28 by using the trained model 116. After the execution of the processing of step ST14, the endoscopic image processing proceeds to step ST16.

In step ST16, the lumen direction estimation unit 58A determines whether or not the estimation of the lumen direction has ended. In step ST16, in a case in which the estimation of the lumen direction has not ended, a negative determination is made, and the endoscopic image processing returns to step ST16 again. In a case in which the estimation of the lumen direction has ended in step ST16, an affirmative determination is made, and the endoscopic image processing proceeds to step ST18.

In step ST18, the information generation unit 58B generates the lumen direction information 120 based on the estimation result 118 obtained in step ST16. After the execution of the processing of step ST18, the endoscopic image processing proceeds to step ST20.

In step ST20, the display control unit 58C outputs the lumen direction information 120 generated in step ST18 to the display 74. After the execution of the processing of step ST20, the endoscopic image processing proceeds to step ST22.

In step ST22, the display control unit 58C determines whether or not a condition for ending the endoscopic image processing (hereinafter, referred to as an “end condition”) is satisfied. Examples of the end condition include a condition in which an instruction to end the endoscopic image processing is received by the touch panel 54. In step ST22, in a case in which the end condition is not satisfied, a negative determination is made, and the endoscopic image processing proceeds to step ST12. In step ST22, in a case in which the end condition is satisfied, an affirmative determination is made, and the endoscopic image processing ends.

It should be noted that, in step ST10, although the form example has been described in which, as the lumen direction estimation start trigger, it is determined whether or not the lumen direction estimation start instruction (for example, the operation of the button (not shown) provided in the endoscope 18) issued by the user is received, the technology of the present disclosure is not limited to this. The lumen direction estimation start trigger may be whether or not it is detected that the endoscope 18 is inserted into the tubular organ. In a case in which it is detected that the endoscope 18 is inserted, the lumen direction estimation start trigger is turned on. In such a case, the processor 58 detects whether or not the endoscope 18 is inserted into the tubular organ by executing, for example, image recognition processing using AI on the endoscopic image 28. Further, another lumen direction estimation start trigger may be whether or not a specific part in the tubular organ is recognized. In a case in which the specific part is detected, the lumen direction estimation start trigger is turned on. Even in such a case, the processor 58 detects whether or not the specific part is detected by executing, for example, image recognition processing using AI on the endoscopic image 28.

In addition, in step ST22, the form example has been described in which the end condition is the condition in which the instruction to end the endoscopic image processing is received by the touch panel 54, but the technology of the present disclosure is not limited to this. For example, the end condition may be a condition in which the processor 58 detects that the endoscope 18 is pulled out from the body. In such a case, the processor 58 detects that the endoscope 18 is pulled out from the body by executing, for example, image recognition processing using AI on the endoscopic image 28. In addition, as another end condition, a condition may be used in which the processor 58 detects that the endoscope 18 has reached the specific part in the tubular organ (for example, an ileocecal part in the large intestine). In such a case, the processor 58 detects that the endoscope 18 has reached the specific part of the tubular organ by executing, for example, image recognition processing using AI on the endoscopic image 28.

As described above, in the endoscope device 12 according to the present embodiment, the lumen direction is acquired by inputting the endoscopic image 28 captured by the camera 38 to the trained model 116. The trained model 116 is obtained through the machine learning processing based on the positional relationship between the plurality of division regions 96 obtained by dividing the image showing the tubular organ (for example, the large intestine) and the lumen corresponding region 94 included in the endoscopic image 28. Further, the processor 58 outputs the lumen direction information 120 that is the information indicating the lumen direction. Therefore, with the present configuration, accurate output of the lumen direction information 120 is implemented. The lumen direction information 120 is used, for example, for the display indicating the lumen direction with respect to the user.

For example, with the present configuration, even in a case in which an image in a state in which the accuracy of prediction is decreased according to an empirical rule (for example, an image in which halation does not occur in the image) is used, the prediction of the lumen direction can be executed, as compared with the prediction of the lumen direction via the image processing in which an empirical prediction in the lumen direction executed by the doctor during the medical examination is applied (for example, the lumen direction is predicted from an arc shape of the halation). Therefore, with the present configuration, accurate output of the lumen direction information 120 is implemented.

In addition, in the endoscope device 12 according to the present embodiment, the predetermined range including the lumen region 28A in the endoscopic image 28 is the lumen corresponding region 94. The lumen direction is estimated in accordance with the trained model 116 obtained through the machine learning based on the positional relationship between the division regions 96 and the lumen corresponding region 94. Since the predetermined range is set as the lumen corresponding region 94, it is easy to recognize the presence of the lumen region 28A in the machine learning, and the accuracy of the machine learning is improved. Therefore, the accuracy of the estimation of the lumen direction using the trained model 116 is also improved. As a result, the lumen direction information 120 having high accuracy is output by the processor 58. Therefore, with the present configuration, accurate output of the lumen direction information 120 is implemented.

For example, in a case in which only the lumen region 28A is set as the lumen corresponding region 94, the lumen corresponding region 94 is small like a point in the image, the lumen corresponding region 94 is not accurately recognized in the machine learning, and the accuracy of the machine learning is decreased. On the other hand, with the present configuration, since the lumen corresponding region 94 is set to the predetermined range, the accuracy of the machine learning is improved. As a result, the lumen direction information 120 having high accuracy is output by the processor 58. Therefore, with the present configuration, accurate output of the lumen direction information 120 is implemented.

In addition, in the endoscope device 12 according to the present embodiment, the end part of the observation range of the camera 38 in a direction in which a position of the lumen is estimated from the fold region 28B in the endoscopic image 28 is the lumen corresponding region 94. The lumen direction is estimated in accordance with the trained model 116 obtained through the machine learning based on the positional relationship between the division regions 96 and the lumen corresponding region 94. Since the end part of the observation range of the camera 38 in the direction in which the position of the lumen is estimated from the fold region 28B is the lumen corresponding region 94, the machine learning can be executed even in a case in which the lumen region 28A is not included in the image. As a result, the number of endoscopic images 28 as learning targets is increased, so that the accuracy of the machine learning is improved. Therefore, the accuracy of the estimation of the lumen direction using the trained model 116 is also improved. As a result, the lumen direction information 120 having high accuracy is output by the processor 58. Therefore, with the present configuration, accurate output of the lumen direction information 120 is implemented.

In addition, in the endoscope device 12 according to the present embodiment, in the positional relationship between the lumen corresponding region 94 and the division regions 96 in the machine learning, the direction of the division region 96 overlapping the lumen corresponding region 94 is the lumen direction. The direction of the division region 96 is determined in advance by the division of the endoscopic image 28. Therefore, with the present configuration, the load in the estimation of the lumen direction is reduced as compared with a case in which the lumen direction is calculated each time in accordance with the position of the lumen corresponding region 94.

In addition, in the endoscope device 12 according to the present embodiment, the trained model 116 is a data structure configured to cause the processor 58 to estimate the position of the lumen based on a shape and/or an orientation of the fold region 28B. Therefore, the position of the lumen is accurately estimated. Therefore, with the present configuration, accurate output of the lumen direction information 120 is implemented.

For example, with the present configuration, even in a case in which an image in a state in which the accuracy of prediction is decreased according to the empirical rule (for example, an image in which halation does not occur in the image) is used, the prediction of the lumen direction can be executed, as compared with the prediction of the lumen direction via the image processing in which the empirical prediction in the lumen direction executed by the doctor during the medical examination is applied (for example, the lumen direction is predicted from an arc shape of the halation). Therefore, with the present configuration, accurate output of the lumen direction information 120 is implemented.

In addition, in the endoscope device 12 according to the present embodiment, the lumen direction is a direction in which the division region 96 having the largest area overlapping the lumen corresponding region 94 is present. The large area in which the lumen corresponding region 94 and the division region 96 overlap each other means that the lumen is present in the direction in which the division region 96 is present. Therefore, in the machine learning, the lumen direction can be uniquely determined. Therefore, with the present configuration, accurate output of the lumen direction information 120 is implemented.

In addition, in the endoscope device 12 according to the present embodiment, the division regions 96 include the central region 96A of the endoscopic image 28 and the plurality of radial regions 96B present radially from the central region 96A toward the outer edge of the endoscopic image 28. In the central region 96A, the lumen region 28A is captured relatively frequently in the endoscopic image 28. Therefore, it is necessary to indicate the lumen direction even in a case in which the lumen is present in the central region 96A. By dividing the endoscopic image 28 radially, it is easy to indicate in which direction the lumen direction is present. By dividing the endoscopic image 28 into the central region 96A and the radial regions 96B in this way, it is easy to understand in which direction the lumen direction is present. Therefore, with the present configuration, it is possible to indicate the lumen direction to the user in an easily understandable manner.

In addition, in the endoscope device 12 according to the present embodiment, the eight radial regions 96B are present radially. Since there are the eight radial regions 96B, it is easy to indicate in which direction the lumen direction is present. In addition, the lumen direction is also indicated to the user in a division that is not too fine. Therefore, with the present configuration, it is possible to indicate the lumen direction to the user in an easily understandable manner.

In addition, in the endoscope device 12 according to the present embodiment, the information corresponding to the lumen direction information 120 output by the processor 58 is displayed on the display device 22. Therefore, with the present configuration, it is easy for the user to recognize the lumen direction.

In addition, the trained model 116 according to the present embodiment is obtained through the machine learning processing based on the positional relationship between the plurality of division regions 96 obtained by dividing the endoscopic image 28 and the lumen corresponding region 94 included in the endoscopic image 28. The trained model 116 is used for the output of the lumen direction information 120 via the processor 58. Therefore, with the present configuration, accurate output of the lumen direction information 120 is implemented. The lumen direction information 120 is used, for example, for the display indicating the lumen direction with respect to the doctor.

For example, with the present configuration, even in a case in which an image in a state in which the accuracy of prediction is decreased according to the empirical rule (for example, an image in which halation does not occur in the image) is used, the prediction of the lumen direction can be executed, as compared with the prediction of the lumen direction via the endoscopic image processing in which the empirical prediction in the lumen direction executed by the doctor during the medical examination is applied (for example, the lumen direction is predicted from an arc shape of the halation). Therefore, with the present configuration, accurate output of the lumen direction information 120 is implemented.

### Second Embodiment

In the first embodiment, the form example has been described in which the direction of the division region 96 having the largest area overlapping the lumen corresponding region 94 is generated as the ground truth data 92 by the operation unit 86, but the technology of the present disclosure is not limited to this. In the second embodiment, the direction of the division region 96 having the largest area overlapping the lumen corresponding region 94 and a direction of the division region 96 having a second largest area overlapping the lumen corresponding region are generated as the ground truth data 92 by the operation unit 86.

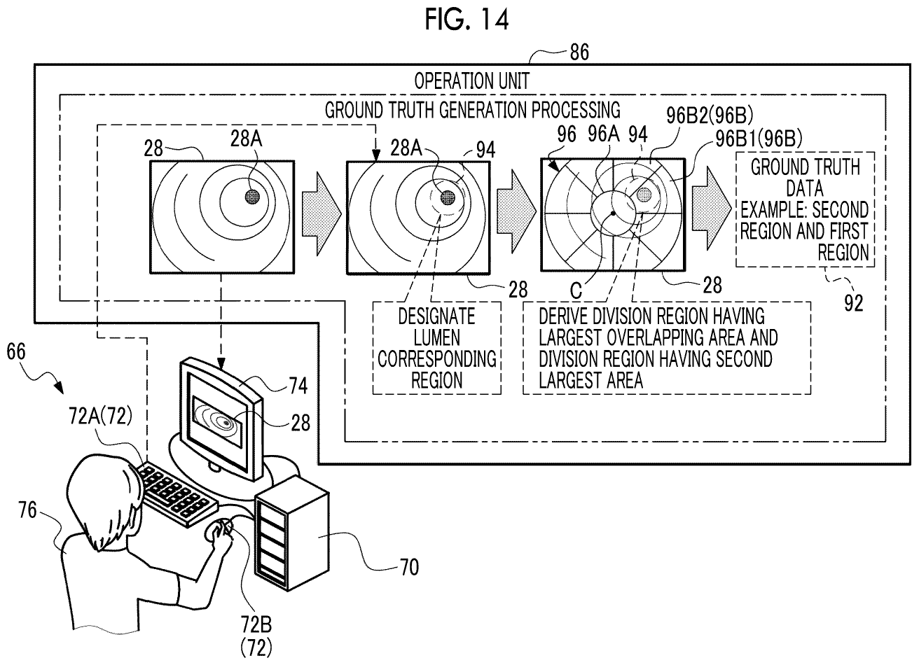

As shown in FIG. 14 as an example, first, the operation unit 86 displays the endoscopic image 28 on the display 74. In a state in which the endoscopic image 28 is displayed on the display 74, the annotator 76 designates the lumen corresponding region 94 in the endoscopic image 28 to the computer 70 via the reception device 72 (for example, the keyboard 72A and/or the mouse 72B).

The operation unit 86 receives the designation of the lumen corresponding region 94 in the endoscopic image 28 from the annotator 76 via the reception device 72. The plurality of division regions 96 are obtained by virtually dividing the endoscopic image 28 via the operation unit 86. In the example shown in FIG. 14, the endoscopic image 28 is divided into the central region 96A and the eight radial regions 96B.

The operation unit 86 derives the division region 96 having the largest area overlapping the lumen corresponding region 94 and the division region 96 having the second largest area overlapping the lumen corresponding region 94 among the plurality of division regions 96. For example, the operation unit 86 specifies the region in which each of the plurality of division regions 96 and the lumen corresponding region 94 overlap each other. In addition, the operation unit 86 calculates the area of the region in which the division region 96 and the lumen corresponding region 94 overlap each other. Then, the operation unit 86 specifies the division region 96 having the largest area and the division region 96 having the second largest area among the regions in which the division region 96 and the lumen corresponding region 94 overlap each other. The division region 96 having the largest area of the region in which the division region 96 and the lumen corresponding region 94 overlap each other is an example of a “first division region” according to the technology of the present disclosure, and the division region 96 having the second largest area is an example of a “second division region” according to the technology of the present disclosure.

In the example shown in FIG. 14, as the ground truth data 92, an example is shown in which a direction in which the second region 96B1 and a first region 96B2 are present in the radial region 96B is the lumen direction (that is, the direction in which the camera 38 is inserted).

Here, although the form example has been described in which the lumen region 28A is captured in the endoscopic image 28, the technology of the present disclosure is not limited to this. For example, as in FIG. 7, the lumen region 28A may not be included in the endoscopic image 28.

The training data generation unit 88 (see FIG. 8) acquires the endoscopic image 28 from the operation unit 86 (see FIG. 8) and associates the ground truth data 92 with the acquired endoscopic image 28 to generate the training data 95 (see FIG. 8). The learning execution unit 90 acquires the training data 95 generated by the training data generation unit 88. The learning execution unit 90 (see FIG. 8) executes the machine learning using the training data 95. A trained model 116A generated as a result of the machine learning is stored in the NVM 62 of the endoscope device 12 as the storage device by the learning execution unit 90.

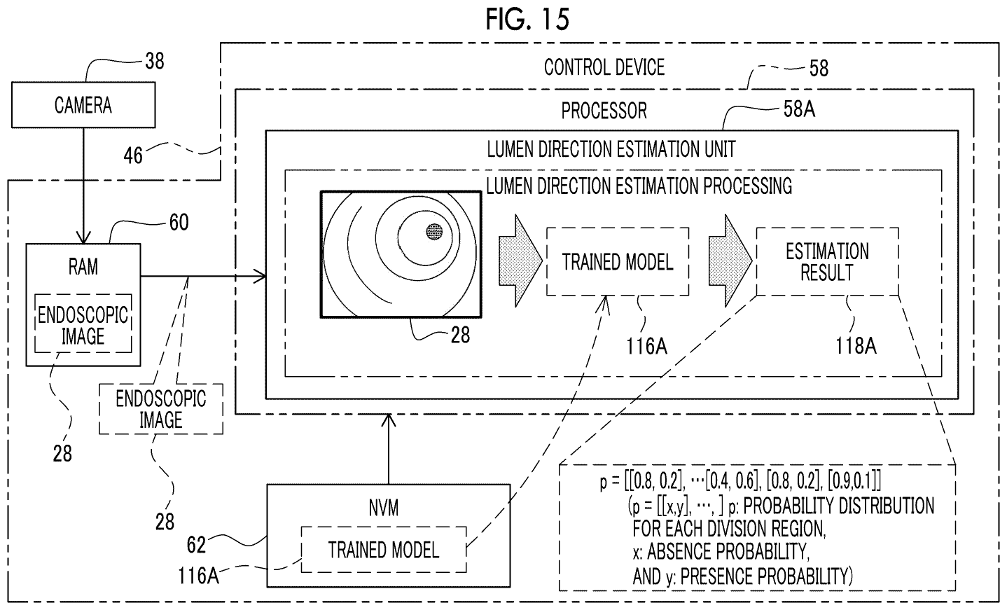

As shown in FIG. 15 as an example, in the endoscope device 12, the lumen direction estimation processing is executed by using the trained model 116A generated in the information processing device 66. The lumen direction estimation unit 58A executes the lumen direction estimation processing based on the endoscopic image 28. The lumen direction estimation unit 58A acquires the trained model 116A from the NVM 62. The lumen direction estimation unit 58A inputs the endoscopic image 28 to the trained model 116A. In a case in which the endoscopic image 28 is input, the trained model 116A outputs an estimation result 118A of the lumen direction in the endoscopic image 28. The estimation result 118A is, for example, the probability distribution p of whether or not the lumen direction is present for each division region 96.

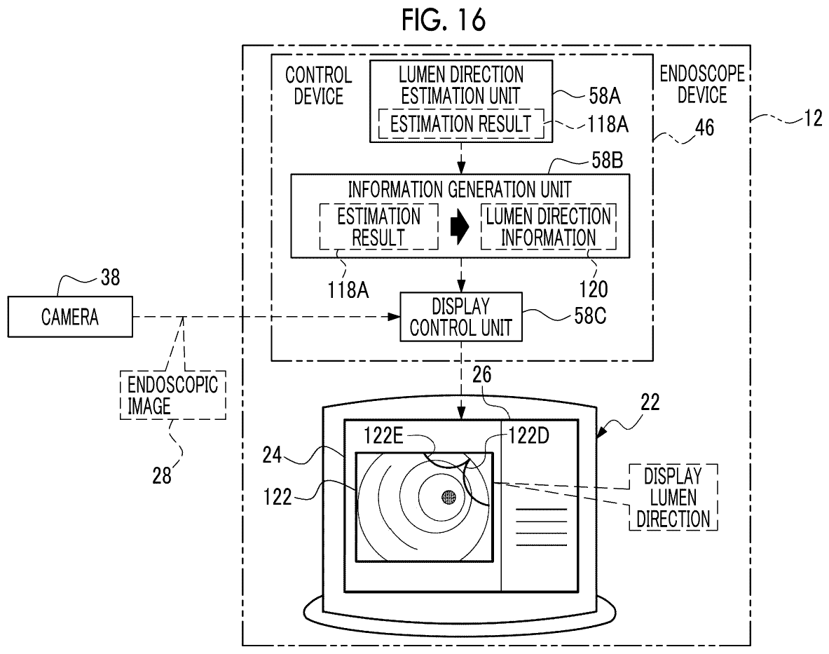

As shown in FIG. 16 as an example, the lumen direction estimation unit 58A outputs the estimation result 118 to the information generation unit 58B. The information generation unit 58B generates the lumen direction information 120 based on the estimation result 118A. The information generation unit 58B generates the lumen direction information 120 by, for example, using the direction of the division region 96 indicating a highest probability distribution value in the probability distribution p indicated by the estimation result 118A and the direction of the division region 96 having a second highest probability distribution value as the lumen directions. The information generation unit 58B outputs the lumen direction information 120 to the display control unit 58C.

The display control unit 58C generates the image 122 in which the lumen direction indicated by the lumen direction information 120 is superimposed and displayed on the endoscopic image 28. The display control unit 58C causes the display device 22 to display the image 122. In the example shown in FIG. 16, in the image 122, an arc 122D and an arc 122E are shown on the outer periphery of the observation range of the endoscopic image 28 as the display indicating the lumen direction.

As described above, in the endoscope device 12 according to the present embodiment, the lumen direction is the direction in which the division region 96 having the largest area overlapping the lumen corresponding region 94 is present and the direction in which the division region 96 having the second largest area overlapping the lumen corresponding region 94 is present. The large area in which the lumen corresponding region 94 and the division region 96 overlap each other means that there is a high probability that the lumen is present in the direction in which the division region 96 is present. As a result, in the machine learning, it is possible to determine a direction having a high probability in which the lumen direction is present. Therefore, with the present configuration, the output of the lumen direction information 120 having a high probability in which the lumen direction is present is implemented.

### First Modification Example

It should be noted that, in the second embodiment, although the form example has been described in which the estimation result 118A output from the trained model 116A is used as it is to generate the lumen direction information 120, the technology of the present disclosure is not limited to this. A correction result 124, which is a result of correcting the estimation result 118A, may be used to generate the lumen direction information 120.

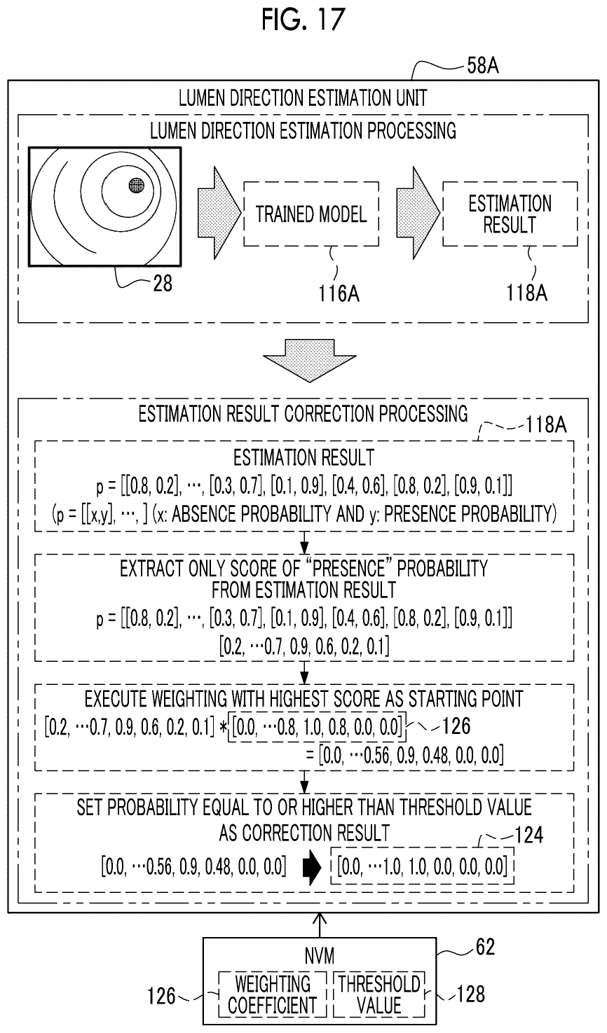

As shown in FIG. 17 as an example, the lumen direction estimation unit 58A executes the lumen direction estimation processing based on the endoscopic image 28. The lumen direction estimation unit 58A inputs the endoscopic image 28 to the trained model 116A. In a case in which the endoscopic image 28 is input, the trained model 116A outputs an estimation result 118A of the lumen direction in the endoscopic image 28.

The lumen direction estimation unit 58A executes estimation result correction processing on the estimation result 118A. The lumen direction estimation unit 58A extracts only the probability in which the lumen direction is present from the probability distribution p of each division region 96 of the estimation result 118A. Further, the lumen direction estimation unit 58A executes weighting with the highest probability in the probability distribution p as a starting point. Specifically, the lumen direction estimation unit 58A acquires a weighting coefficient 126 from the NVM 62 and multiplies the extracted probability by the weighting coefficient 126. The weighting coefficient 126 is set, for example, such that a coefficient corresponding to the highest probability is 1 and a coefficient corresponding to a probability adjacent to the highest probability is 0.8. The weighting coefficient 126 is appropriately set based on, for example, the past estimation result 118A.

The weighting coefficient 126 may be set in accordance with the probability distribution p. For example, in a case in which the probability of the central region 96A in the division region 96 is the highest, a coefficient corresponding to the highest probability among the weighting coefficients 126 may be set to 1, and the coefficients other than the coefficient corresponding to the highest probability may be set to 0.

Then, the lumen direction estimation unit 58A acquires a threshold value 128 from the NVM 62, and sets the probability equal to or higher than the threshold value 128 as the correction result 124. The threshold value 128 is, for example, 0.5, but this is merely an example. The threshold value 128 may be, for example, 0.4 or 0.6. The threshold value 128 is appropriately set based on, for example, the past estimation result 118A.

The lumen direction estimation unit 58A outputs the correction result 124 to the information generation unit 58B. The information generation unit 58B generates the lumen direction information 120 based on the correction result 124. The information generation unit 58B outputs the lumen direction information 120 to the display control unit 58C.

As described above, in the endoscope device 12 according to the first modification example, the estimation result 118A is corrected by the estimation result correction processing. In the estimation result correction processing, the weighting coefficient 126 and the threshold value 128 are used to correct the estimation result 118A. Therefore, the lumen direction indicated by the estimation result 118A is more accurate. Therefore, with the present configuration, accurate output of the lumen direction information 120 is implemented.

It should be noted that, in the first modification example, although the form example has been described in which the estimation result correction processing is executed on the estimation result 118A, the technology of the present disclosure is not limited to this. The operation corresponding to the estimation result correction processing may be incorporated in the trained model 116A.

### Second Modification Example

In the first and second embodiments, the form example has been described in which the division regions 96 include the central region 96A and the radial regions 96B, but the technology of the present disclosure is not limited to this. In the second modification example, the division regions 96 include the central region 96A and a plurality of peripheral regions 96C present on an outer edge side of the endoscopic image 28 with respect to the central region 96A.

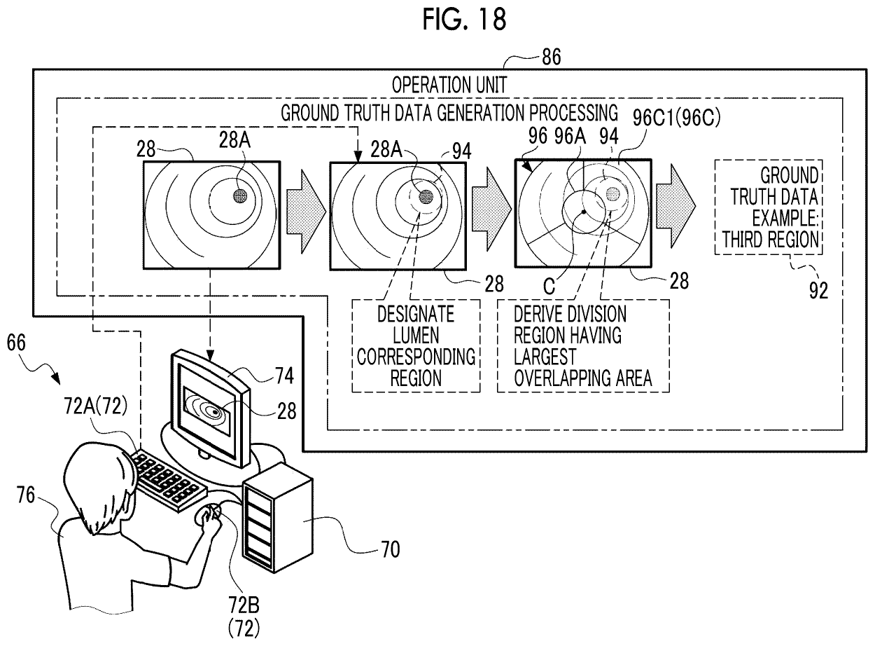

As shown in FIG. 18 as an example, the operation unit 86 receives the designation of the lumen corresponding region 94 in the endoscopic image 28 from the annotator 76 via the reception device 72. The plurality of division regions 96 are obtained by virtually dividing the endoscopic image 28 via the operation unit 86.

The division regions 96 include the central region 96A and the peripheral regions 96C. The central region 96A is, for example, the circular region centered on the center C in the endoscopic image 28. The peripheral regions 96C are a plurality of regions present on the outer edge side of the endoscopic image 28 with respect to the central region 96A. In the example shown in FIG. 18, three peripheral regions 96C are present on the outer edge side of the endoscopic image 28. Here, the three peripheral regions 96C are shown, but this is merely an example. The number of peripheral regions 96C may be two or four or more. The peripheral region 96C is an example of a “peripheral region” according to the technology of the present disclosure.

The operation unit 86 derives the division region 96 having the largest area overlapping the lumen corresponding region 94 among the plurality of division regions 96. For example, the operation unit 86 specifies the region in which each of the plurality of division regions 96 and the lumen corresponding region 94 overlap each other. The operation unit 86 calculates the area of the region in which the division region 96 and the lumen corresponding region 94 overlap each other. Then, the operation unit 86 specifies the division region 96 having the largest area of the region in which the division region 96 and the lumen corresponding region 94 overlap each other.

The operation unit 86 generates the direction of the division region 96 having the largest area overlapping the lumen corresponding region 94 as the ground truth data 92. In the example shown in FIG. 18, an example is shown in which a direction in which a third region 96C1 is present among the peripheral regions 96C is the lumen direction, as the ground truth data 92.

As described above, in the second modification example, the division regions 96 include the central region 96A of the endoscopic image 28 and the plurality of peripheral regions 96C present on the outer edge side of the endoscopic image 28 with respect to the central region 96A. In the central region 96A, the lumen region 28A is captured relatively frequently in the endoscopic image 28. Therefore, it is necessary to indicate the lumen direction even in a case in which the lumen is present in the central region 96A. By dividing the peripheral region 96C into a plurality of regions, it is easy to indicate in which direction the lumen direction is present. By dividing the endoscopic image 28 into the central region 96A and the plurality of peripheral regions 96C in this way, it is easy to understand in which direction the lumen direction is present. Therefore, with the present configuration, it is possible to indicate the lumen direction to the user in an easily understandable manner.

In addition, in the second modification example, among the division regions 96, the peripheral regions 96C are obtained by dividing the outer edge side of the endoscopic image 28 with respect to the central region 96A into three or more directions from the central region 96A toward the outer edge of the endoscopic image 28. In the central region 96A, the lumen region 28A is captured relatively frequently in the endoscopic image 28. Therefore, it is necessary to indicate the lumen direction even in a case in which the lumen is present in the central region 96A. By dividing the endoscopic image 28 into three or more directions toward the outer edge, it is easy to understand in which direction the lumen direction is present. Through the division into the central region 96A and the peripheral regions 96C in three or more directions in this way, it is easy to understand in which direction the lumen direction is present. Therefore, with the present configuration, it is possible to indicate the lumen direction to the user in an easily understandable manner.

### Third Modification Example

In the first and second embodiments, although the form example has been described in which the division regions 96 include the central region 96A and the radial regions 96B, the technology of the present disclosure is not limited to this. In the third modification example, the division regions 96 are obtained by dividing the endoscopic image 28 into regions in three or more directions toward the outer edge of the endoscopic image 28 with the center C of the endoscopic image 28 as a starting point.

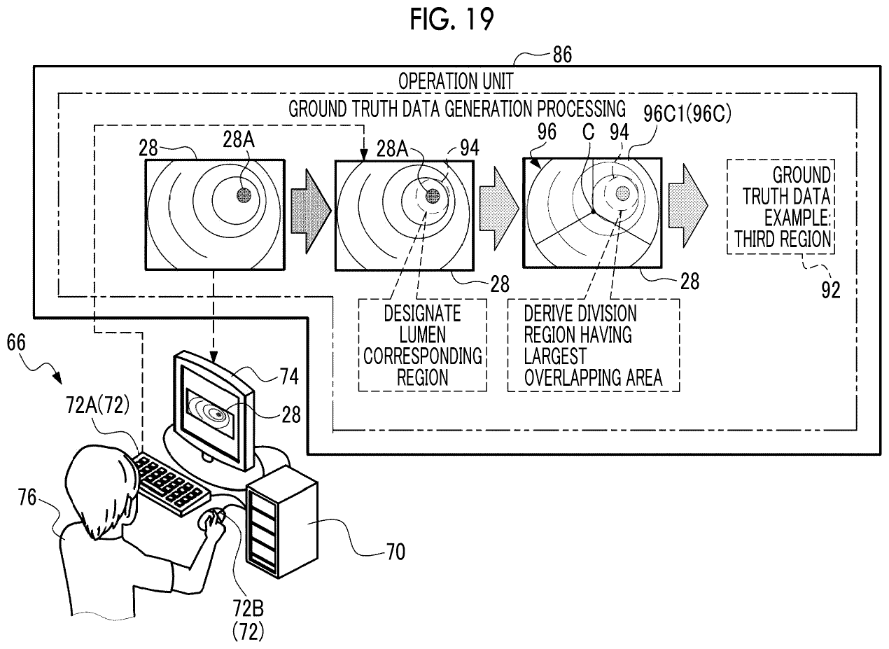

As shown in FIG. 19 as an example, the operation unit 86 receives the designation of the lumen corresponding region 94 in the endoscopic image 28 from the annotator 76 via the reception device 72. The plurality of division regions 96 are obtained by virtually dividing the endoscopic image 28 via the operation unit 86.

The division regions 96 are regions obtained by dividing the endoscopic image 28 in three directions toward the outer edge of the endoscopic image 28 with the center C of the endoscopic image 28 as a center. In the example shown in FIG. 19, three division regions 96 are present on the outer edge side of the endoscopic image 28. Here, the three division regions 96 are shown, but this is merely an example. The number of division regions 96 may be two or four or more.

The operation unit 86 derives the division region 96 having the largest area overlapping the lumen corresponding region 94 among the plurality of division regions 96. For example, the operation unit 86 specifies the region in which each of the plurality of division regions 96 and the lumen corresponding region 94 overlap each other. The operation unit 86 calculates the area of the region in which the division region 96 and the lumen corresponding region 94 overlap each other. Then, the operation unit 86 specifies the division region 96 having the largest area of the region in which the division region 96 and the lumen corresponding region 94 overlap each other.

The operation unit 86 generates the direction of the division region 96 having the largest area overlapping the lumen corresponding region 94 as the ground truth data 92. In the example shown in FIG. 19, an example is shown in which the direction in which the third region 96C1 is present among the peripheral regions 96C is the lumen direction, as the ground truth data 92.

As described above, in the third modification example, the division region 96 is obtained by dividing the endoscopic image 28 in three or more directions with the center C of the endoscopic image 28 as a starting point toward the outer edge.

By dividing the endoscopic image 28 into three or more directions with the center C as a starting point toward the outer edge, it is easy to understand in which direction the lumen direction is present. By dividing the region into three or more directions in this way, it is easy to understand in which direction the lumen direction is present. Therefore, with the present configuration, it is possible to indicate the lumen direction to the user in an easily understandable manner.

In each of the above-described embodiments, the form example has been described in which the endoscopic image processing is executed by the processor 58 of the endoscope device 12, but the technology of the present disclosure is not limited to this. For example, the device that executes the endoscopic image processing may be provided outside the endoscope device 12. Examples of the device provided outside the endoscope device 12 include a server. For example, the server is implemented by cloud computing. Here, cloud computing has been described as an example, but this is merely an example, and, for example, the server may be implemented by a mainframe or may be implemented by network computing such as fog computing, edge computing, or grid computing. Here, the server has been described as the device provided outside the endoscope device 12, but this is merely an example, and, for example, at least one personal computer may be used instead of the server. In addition, the endoscopic image processing may be distributedly executed by a plurality of devices including the endoscope device 12 and a device provided outside the endoscope device 12.

In each of the above-described embodiments, the form example has been described in which the endoscopic image processing program 62A is stored in the NVM 62, but the technology of the present disclosure is not limited to this. For example, the endoscopic image processing program 62A may be stored in a portable storage medium, such as an SSD or a USB memory. The storage medium is a non-transitory computer-readable storage medium. The endoscopic image processing program 62A stored in the storage medium is installed in the computer 56 of the control device 46. The processor 58 executes the endoscopic image processing in accordance with the endoscopic image processing program 62A.

In addition, in each of the above-described embodiments, the form example has been described in which the machine learning processing is executed by the processor 78 of the information processing device 66, but the technology of the present disclosure is not limited to this. For example, the machine learning processing may be executed in the endoscope device 12. In addition, the machine learning processing may be executed in a distributed manner by a plurality of devices including the endoscope device 12 and the information processing device 66.

Further, in each of the above-described embodiments, the form example has been described in which the lumen direction is displayed based on the estimation result 118 obtained by inputting the endoscopic image 28 to the trained model 116, but the technology of the present disclosure is not limited to this. For example, the lumen direction may be displayed by using the estimation result 118 for another endoscopic image 28 (for example, an endoscopic image 28 obtained a few frames (for example, 1 to 2 frames) before the endoscopic image 28) in combination with the estimation result 118 for the endoscopic image 28.

In each of the above-described embodiments, the computer 56 is described as an example, but the technology of the present disclosure is not limited to this, and a device including an ASIC, an FPGA, and/or a PLD may be applied instead of the computer 56. Further, a combination of a hardware configuration and a software configuration may be used instead of the computer 56.

The following various processors can be used as hardware resources for executing each of the various kinds of processing described in each of the above-described embodiments. Examples of the processor include a CPU which is a general-purpose processor functioning as the hardware resource for executing the endoscopic image processing by executing software, that is, a program. Examples of the processor also include a dedicated electronic circuit as a processor having a dedicated circuit configuration specially designed to execute specific processing, such as an FPGA, a PLD, or an ASIC. Any processor has a memory built in or connected to it, and any processor executes the endoscopic image processing by using the memory.

The hardware resource for executing the endoscopic image processing may be configured by one of the various processors or by a combination of two or more processors of the same type or different types (for example, a combination of a plurality of FPGAs or a combination of a processor and an FPGA). Further, the hardware resource for executing the endoscopic image processing may be one processor.

A first example of the configuration in which the hardware resource is configured by one processor is an aspect in which one processor is configured by a combination of one or more processors and software, and this processor functions as the hardware resource for executing the endoscopic image processing. Secondly, as represented by an SoC, there is a form in which a processor that realizes the functions of the entire system including a plurality of hardware resources for executing the endoscopic image processing with one IC chip is used. As described above, the endoscopic image processing is implemented by using one or more of the various processors as the hardware resources.

Further, specifically, an electronic circuit obtained by combining circuit elements, such as semiconductor elements, can be used as the hardware structure of these various processors. The endoscopic image processing is merely an example. Therefore, it goes without saying that unnecessary steps may be deleted, new steps may be added, or the processing order may be changed within a range that does not deviate from the gist.

The above-described contents and the above-shown contents are the detailed description of the parts according to the technology of the present disclosure, and are merely examples of the technology of the present disclosure. For example, the description of the configuration, the function, the operation, and the effect are the description of examples of the configuration, the function, the operation, and the effect of the parts according to the technology of the present disclosure. Accordingly, it goes without saying that unnecessary parts may be deleted, new elements may be added, or replacements may be made with respect to the above-described contents and the above-shown contents within a range that does not deviate from the gist of the technology of the present disclosure. In addition, in order to avoid complications and facilitate understanding of the parts according to the technology of the present disclosure, the description of common technical knowledge or the like, which does not particularly require the description for enabling the implementation of the technology of the present disclosure, is omitted in the above-described contents and the above-shown contents.

In the present specification, “A and/or B” is synonymous with “at least one of A or B”. That is, “A and/or B” may mean only A, only B, or a combination of A and B. In the present specification, the same concept as “A and/or B” also applies to a case in which three or more matters are expressed by association with “and/or”.

All of the documents, the patent applications, and the technical standards described in the present specification are incorporated into the present specification by reference to the same extent as in a case in which the individual documents, patent applications, and technical standards are specifically and individually stated to be described by reference.

The disclosure of JP2022-115110 filed on Jul. 19, 2022 is incorporated in the present specification by reference in its entirety.

What is claimed is:

1. An image processing device comprising:
a processor,
wherein the processor is configured to:
acquire a lumen direction that is a direction in which an endoscope is inserted, from an image obtained by imaging a tubular organ via a camera provided in the endoscope, in accordance with a trained model obtained through machine learning based on a positional relationship between a plurality of division regions obtained by dividing the image and a lumen corresponding region included in the image, and
output lumen direction information that is information indicating the lumen direction.

2. The image processing device according to claim 1,
wherein the lumen corresponding region is a region in a predetermined range including a lumen region in the image.

3. The image processing device according to claim 1,
wherein the lumen corresponding region is an end part of an observation range of the camera in a direction in which a position of the lumen region is estimated from a fold region in the image.

4. The image processing device according to claim 1,
wherein a direction of a division region overlapping the lumen corresponding region among the plurality of division regions is the lumen direction.

5. The image processing device according to claim 1,
wherein the trained model is a data structure configured to cause the processor to estimate a position of the lumen region based on a shape and/or an orientation of a fold region in the image.

6. The image processing device according to claim 1,
wherein the lumen direction is a direction in which a division region having a largest area overlapping the lumen corresponding region in the image among the plurality of division regions is present.

7. The image processing device according to claim 1,
wherein the lumen direction is a direction in which, among the plurality of division regions, a first division region that is a division region having a largest area overlapping the lumen corresponding region in the image is present and a direction in which a second division region that is a division region having a second largest area overlapping the lumen corresponding region following the first division region is present.

8. The image processing device according to claim 1,
wherein the division regions include a central region of the image and a plurality of radial regions that are present radially from the central region toward an outer edge of the image.

9. The image processing device according to claim 8,
wherein eight radial regions are present radially.

10. The image processing device according to claim 1,
wherein the division regions include a central region of the image and a plurality of peripheral regions present on an outer edge side of the image with respect to the central region.

11. The image processing device according to claim 1,
wherein the division regions are obtained by dividing the image into regions in three or more directions toward an outer edge of the image with a center of the image as a starting point.

12. The image processing device according to claim 1,
wherein the division regions include a central region of the image and a plurality of peripheral regions present on an outer edge side of the image with respect to the central region, and
the peripheral regions are obtained by dividing the outer edge side of the image with respect to the central region in three or more directions from the central region toward an outer edge of the image.

13. A display device that displays information corresponding to the lumen direction information output by the processor of the image processing device according to claim 1.

14. An endoscope device comprising:
the image processing device according to any one of claim 1; and
the endoscope.

15. An image processing method comprising:
acquiring a lumen direction that is a direction in which an endoscope is inserted, from an image obtained by imaging a tubular organ via a camera provided in the endoscope, in accordance with a trained model obtained through machine learning based on a positional relationship between a plurality of division regions obtained by dividing the image and a lumen corresponding region included in the image; and
outputting lumen direction information that is information indicating the lumen direction.

16. A non-transitory computer-readable storage medium storing an image processing program executable by a first computer to execute image processing comprising:
acquiring a lumen direction that is a direction in which an endoscope is inserted, from an image obtained by imaging a tubular organ via a camera provided in the endoscope, in accordance with a trained model obtained through machine learning based on a positional relationship between a plurality of division regions obtained by dividing the image and a lumen corresponding region included in the image; and
outputting lumen direction information that is information indicating the lumen direction.
